SEQUENCE LISTING (7 sequences)

SEQ ID NO: 1 - DNA, 17 bp, synthetic construct
  misc_feature 1..17: note = FP1 forward
  source 1..17: mol_type = other DNA
actcaacaaggaagtga

SEQ ID NO: 2 - DNA, 17 bp, synthetic construct
  misc_feature 1..17: note = FP2 reverse
  source 1..17: mol_type = other DNA
aattccgcctacctctg

SEQ ID NO: 3 - DNA, 22 bp, synthetic construct
  misc_feature 1..22: note = ERIC-1 forward
  source 1..22: mol_type = other DNA
atgtaagctcctggggattcac

SEQ ID NO: 4 - DNA, 22 bp, synthetic construct
  misc_feature 1..22: note = ERIC-2 reverse
  source 1..22: mol_type = other DNA
aagtaagtgactggggtgagcg

SEQ ID NO: 5 - DNA, 15 bp, synthetic construct
  misc_feature 1..15: note = (GTG)5
  source 1..15: mol_type = other DNA
gtggtggtggtggtg

SEQ ID NO: 6 - DNA, 20 bp, synthetic construct
  misc_feature 1..20: note = 27F
  source 1..20: mol_type = other DNA
agagtttgatcmtggctcag

SEQ ID NO: 7 - DNA, 19 bp, synthetic construct
  misc_feature 1..19: note = 1492R
  source 1..19: mol_type = other DNA
ggttaccttgttacgactt

(19) United States
(12) Patent Application Publication
(10) Pub. No.: US 2025/0099511 A1
(43) Pub. Date: Mar. 27, 2025

(54) PHARMACEUTICAL COMPOSITION FOR PREVENTING OR TREATING CANCER

(71) Applicant: ENTEROBIOME INC., Goyang-si, Gyeonggi-do (KR)

(72) Inventors: Jae-Gu SEO, Gimpo-si (KR); Joo-Hyun SHIN, Seoul (KR); Dokyung LEE, Seoul (KR); Yoonmi LEE, Goyang-si (KR); Seo Yul JANG, Goyang-si (KR); Hye Rim BYEON, Paju-si (KR); Dohak KIM, Gwangmyeong-si (KR); Moon-Gi HONG, Goyang-si (KR)

(73) Assignee: ENTEROBIOME INC., Goyang-si, Gyeonggi-do (KR)

(21) Appl. No.: 18/728,309

(22) PCT Filed: Feb. 15, 2023
(86) PCT No.: PCT/KR2023/002180

§ 371 Date: Jul. 11, 2024

(30) Foreign Application Priority Data
Feb. 15, 2022 (KR) 10-2022-0019423
Jan. 19, 2023 (KR) 10-2023-0007838

(51) Int. Cl. A61K 35/741; A61K 45/06; A61P 35/00

(52) U.S. Cl. CPC A61K 35/741; A61K 45/06; A61P 35/00

(57) ABSTRACT

Compositions containing extracellular vesicles (EVs) derived from a Faecalibacterium sp. strain are disclosed. The compositions can be used to prevent and/or treat cancer and/or ameliorate symptoms of cancer, and can be pharmaceutical composition, food stuffs, or dietary supplements. The compositions may contain a pharmaceutically acceptable carrier or excipient. The compositions exhibit an excellent effect on the prevention or treatment of cancer, or ameliorating symptoms of cancer.

### TECHNICAL FIELD

The present invention relates to a pharmaceutical composition for preventing or treating cancer, and more particularly, to a pharmaceutical composition for preventing or treating cancer that contains extracellular vesicles (EVs) derived from a Faecalibacterium sp. strain.

### BACKGROUND ART

Cancer is a product of uncontrolled and disordered cell proliferation due to an excess of abnormal cells, and a malignant tumor leaves its primary site and invades other tissues, where it grows rapidly. Due to this characteristic, malignant tumors threaten life.

In order to treat cancer, various therapeutic approaches have been attempted, including chemotherapy using various anticancer drugs, radiotherapy, and antibody therapy targeting specific molecules involved in cancer. However, in the case of chemotherapy or radiotherapy, it also affects normal cells, causing serious side effects, and cancer cells often acquire resistance to anticancer drugs, resulting in treatment failure or recurrence.

Recent studies have reported that extracellular vesicles play an important role in processes such as intercellular signaling and waste management. Thus, clinical applications of extracellular vesicles have recently been of increasing interest. It is expected that, based on the characteristics of specific extracellular vesicles and target cell membranes, it will be possible to develop therapeutic agents that can specifically treat only diseased cells, including cancer cells, without causing side effects on other normal cells.

Meanwhile, studies on the effects of combination therapy of probiotics, which exhibit in vivo beneficial effects such as immunity strengthening, with immune checkpoint inhibitors, have been emphasized in terms of the development of pharmabiotics, but studies associated therewith in various cancer types still remain insufficient.

### DISCLOSURE

### Technical Problem

The present invention has been made with the foregoing background in mind, and an object of the present invention is to provide a pharmaceutical composition for preventing or treating cancer that contains pharmabiotics-derived extracellular vesicles.

Another object of the present invention is to provide a health functional food for preventing or ameliorating cancer.

Still another object of the present invention is to provide a veterinary composition or a feed additive for preventing or treating cancer.

Yet another object of the present invention is to provide a method of treating cancer in a patient by activating the immune system of the patient using pharmabiotics-derived extracellular vesicles.

### Technical Solution

One aspect of the present invention for achieving the above-described objects is directed to a pharmaceutical composition for preventing or treating cancer containing: extracellular vesicles (EVs) derived from a Faecalibacterium sp. strain; and a pharmaceutically acceptable carrier or excipient.

The Faecalibacterium sp. strain may be a Faecalibacterium prausnitzii strain, and preferably may be a Faecalibacterium prausnitzii EB-FPDK3 strain (KCCM12619P), an F. prausnitzii EB-FPDK9 strain (KCCM12620P), an F. prausnitzii EB-FPDK11 strain (KCCM12621P), or an F. prausnitzii EB-FPYYK1 strain (KCCM12622P).

The pharmaceutical composition for preventing or treating cancer according to the present invention may further contain a cancer treatment agent such as a cancer chemotherapeutic agent or a cancer immunotherapeutic agent. The cancer immunotherapeutic agent may be selected from the group consisting of anti-PD1, anti-PDL1, anti-CTLA, anti-Tim3, and anti-LAG3. The extracellular vesicles (EVs) derived from the Faecalibacterium sp. strain and the cancer chemotherapeutic agent or cancer immunotherapeutic agent may be administered simultaneously in a single dosage form, or may be administered simultaneously or sequentially in separate dosage forms.

Another aspect of the present invention is directed to a pharmaceutical composition for preventing or treating cancer containing: a Faecalibacterium sp. strain; and a pharmaceutically acceptable carrier or excipient.

Still another aspect of the present invention is directed to a health functional food for preventing or ameliorating cancer containing: extracellular vesicles (EVs) derived from a Faecalibacterium sp. strain; and a physiologically acceptable carrier or excipient.

Yet another aspect of the present invention is directed to a method for treating cancer including administering to a subject a therapeutically effective amount of a Faecalibacterium sp. strain or extracellular vesicles (EVs) derived from the Faecalibacterium sp. strain.

Still yet another aspect of the present invention is directed to a veterinary composition for preventing or treating cancer containing: a Faecalibacterium sp. strain or extracellular vesicles (EVs) derived from the Faecalibacterium sp. strain; and an acceptable carrier or excipient.

The present invention also provides a novel Faecalibacterium prausnitzii EB-FPDK3 strain (KCCM12619P), F. prausnitzii EB-FPDK9 strain (KCCM12620P), F. prausnitzii EB-FPDK11 strain (KCCM12621P), and F. prausnitzii EB-FPYYK1 strain (KCCM12622P).

### Advantageous Effects

The pharmaceutical composition for preventing or treating cancer containing a Faecalibacterium sp. strain or extracellular vesicles (EVs) derived from the Faecalibacterium sp. strain according to the present invention may reduce tumor size, reduce tumor growth, prevent metastasis, or prevent angiogenesis. Thus, it may be developed as an effective anticancer agent.

When the pharmaceutical composition for preventing or treating cancer containing a Faecalibacterium sp. strain or extracellular vesicles (EVs) derived from the Faecalibacterium sp. strain according to the present invention is administered alone, it may exhibit an excellent anticancer effect. In addition, when the pharmaceutical composition is administered in combination with a cancer chemotherapeutic agent or a cancer immunotherapeutic agent, the efficacy thereof may be further activated while the side effects of the cancer chemotherapeutic agent or cancer immunotherapeutic agent are reduced. Thus, co-administration of the pharmaceutical composition and the cancer chemotherapeutic agent or cancer immunotherapeutic agent may exhibit a better anticancer effect compared to when the pharmaceutical composition is administered alone.

### DESCRIPTION OF DRAWINGS

FIG. 1 shows the results of microscopic observation of the Faecalibacterium prausnitzii EB-FPDK3, F. prausnitzii EB-FPDK9, F. prausnitzii EB-FPDK11, and F. prausnitzii EB-FPYYK1 strains of the present invention and the type strain Faecalibacterium prausnitzii A2-165 strain;

FIG. 2 shows the results of PCR analysis of the Faecalibacterium prausnitzii EB-FPDK3, F. prausnitzii EB-FPDK9, F. prausnitzii EB-FPDK11, and F. prausnitzii EB-FPYYK1 strains of the present invention and the type strain Faecalibacterium prausnitzii A2-165 strain;

FIG. 3 shows the results of random amplified polymorphic DNA (RAPD) of the genomic DNA of each of the Faecalibacterium prausnitzii EB-FPDK3, F. prausnitzii EB-FPDK9, F. prausnitzii EB-FPDK11, and F. prausnitzii EB-FPYYK1 strains of the present invention and the type strain Faecalibacterium prausnitzii A2-165 strain;

FIG. 4 shows a phylogenetic tree of the Faecalibacterium prausnitzii EB-FPDK3, F. prausnitzii EB-FPDK9, F. prausnitzii EB-FPDK11, and F. prausnitzii EB-FPYYK1 strains, prepared based on the 16S rRNA sequence;

FIG. 5 shows the result of examining whether the Faecalibacterium prausnitzii EB-FPDK3, F. prausnitzii EB-FPDK9, F. prausnitzii EB-FPDK11, and F. prausnitzii EB-FPYYK1 strains of the present invention and the type strain Faecalibacterium prausnitzii A2-165 strain have hemolytic activity;

FIG. 6 shows electron micrographs of extracellular vesicles derived from each of the Faecalibacterium prausnitzii EB-FPDK3, F. prausnitzii EB-FPDK9, F. prausnitzii EB-FPDK11, and F. prausnitzii EB-FPYYK1 strains of the present invention;

FIGS. 7a and 7b are schematic views showing animal experiment procedures performed in Examples 4 and 5 to evaluate the anticancer activity of each of the Faecalibacterium prausnitzii EB-FPDK3, F. prausnitzii EB-FPDK9, F. prausnitzii EB-FPDK11, and F. prausnitzii EB-FPYYK1 strains of the present invention or the extracellular vesicles (EVs) derived from each of the strains;

FIGS. 8 and 9 show the anti-oncogenic effect of co-administration of each of the Faecalibacterium prausnitzii EB-FPDK3, F. prausnitzii EB-FPDK9, F. prausnitzii EB-FPDK11, and F. prausnitzii EB-FPYYK1 strains of the present invention and aPD-1 in a syngeneic tumor mouse model;

FIG. 10 shows photographs comparing mouse tumor size between experimental groups for 20 days in a syngeneic tumor animal model in order to evaluate the anticarcinogenic effect of co-administration of each of the Faecalibacterium prausnitzii EB-FPDK3, F. prausnitzii EB-FPDK9, F. prausnitzii EB-FPDK11, and F. prausnitzii EB-FPYYK1 strains of the present invention and aPD-1 antibody in Example 4;

FIG. 11 is a graph showing time-dependent changes in tumor size in a syngeneic tumor animal model used to evaluate the anticarcinogenic effect of extracellular vesicles derived from each of the Faecalibacterium prausnitzii EB-FPDK3, F. prausnitzii EB-FPDK9, F. prausnitzii EB-FPDK11, and F. prausnitzii EB-FPYYK1 strains of the present invention in Example 5;

FIG. 12 is a graph showing changes in tumor size and weight for 25 days when extracellular vesicles derived from each of the Faecalibacterium prausnitzii EB-FPDK3, F. prausnitzii EB-FPDK9, F. prausnitzii EB-FPDK11, and F. prausnitzii EB-FPYYK1 strains of the present invention and anti-PD1 were administered in combination to a syngeneic animal model and when anti-PD1 was administered alone to a syngeneic animal model;

FIG. 13 shows photographs comparing mouse tumor size between experimental groups for 25 days in a syngeneic tumor animal model in order to evaluate the anticarcinogenic effect of extracellular vesicles derived from each of the Faecalibacterium prausnitzii EB-FPDK3, F. prausnitzii EB-FPDK9, F. prausnitzii EB-FPDK11, and F. prausnitzii EB-FPYYK1 strains of the present invention;

FIGS. 14a and 14b show the results of evaluating the anticancer activity of extracellular vesicles derived from each of the Faecalibacterium prausnitzii EB-FPDK3, F. prausnitzii EB-FPDK9, F. prausnitzii EB-FPDK11, and F. prausnitzii EB-FPYYK1 strains of the present invention;

FIG. 15 is a schematic diagram showing an animal experiment scheme for evaluating the anticancer effect of administration of Faecalibacterium prausnitzii EB-FPDK9 or EB-FPDK9-derived extracellular vesicles (EB-FPDK9EVs) in a syngeneic melanoma mouse animal model;

FIG. 16 shows a tumor growth curve following administration of the Faecalibacterium prausnitzii EB-FPDK9 strain or EB-FPDK9 EVs; and

FIG. 17 shows photographs comparing tumor size at the end of administration of the Faecalibacterium prausnitzii EB-FPDK9 strain or EB-FPDK9 EVs.

### MODE FOR INVENTION

Hereinafter, the present invention will be described in more detail with reference to the accompanying drawings.

Unless defined otherwise, all technical and scientific terms used herein have the same meaning as commonly understood by one of ordinary skill in the art to which the present invention pertains.

Throughout the present specification, it is to be understood that when any part is referred to as “including,” “comprising” or “containing” any component, it does not exclude other components, but may further include other components, unless otherwise specified.

As used herein, the term “treat,” “treatment” or the like means temporarily or permanently alleviating symptoms, eliminating the cause of symptoms, or preventing or delaying the onset of symptoms of a disease or condition.

As used herein, the term “prevention” refers to any action that suppresses or delays cancer or the onset thereof by administration of the pharmaceutical composition according to the present invention.

As used herein, the term “amelioration” refers to any action that reduces a parameter associated with an abnormal condition, e.g., the severity of a symptom.

As used herein, the term “pharmaceutically acceptable” means that a composition within the scope of reasonable medical judgment, suitable for use in contact with the tissues of a subject (such as a human), without excessive toxicity, irritation, allergic reaction, or other problems or complications, and with a quite reasonable benefit/risk ratio.

As used herein, the term “immune checkpoint inhibitor” refers to a type of drug that blocks certain proteins produced by certain types of cells of the immune system, such as T lymphocytes, and some types of cancer cells, in which these proteins suppress immune response and prevent T lymphocytes from killing cancer cells. “Immune checkpoint inhibitors” well known to date include PD-1/PD-L1 and CTLA-4/B7-1/B7-2.

One aspect of the present invention is directed to a pharmaceutical composition for preventing or treating cancer containing: extracellular vesicles (EVs) derived from a Faecalibacterium sp. strain; and a pharmaceutically acceptable carrier or excipient.

Pharmabiotics are defined as bacterial cells of human origin, or their products, with a proven pharmacological role in health or disease (“Probiotics and pharmabiotics,” Bioeng Bugs. 2010 March-Apr; 1(2): 79-84.). The pharmaceutical composition of the present invention contains pharmabiotics as an active ingredient, and thus may be safely used without side effects.

The Faecalibacterium sp. strain may be a Faecalibacterium prausnitzii strain. Specifically, the Faecalibacterium sp. strain may be a Faecalibacterium prausnitzii EB-FPDK3 strain (KCCM12619P), an F. prausnitzii EB-FPDK9 strain (KCCM12620P), an F. prausnitzii EB-FPDK11 strain (KCCM12621P), or an F. prausnitzii EB-FPYYK1 strain (KCCM12622P).

Extracellular vesicles (EVs) are nano-vesicles that are secreted from cells. These extracellular vesicles contain immunologically important proteins such as the main histocompatibility complex (MHC) and heat shock protein, which induce a strong antitumor immune response. In addition, they contain anti-inflammatory microRNA and microRNA that regulates collagen accumulation.

The Faecalibacterium sp. strain-derived extracellular vesicles (EVs) of the present invention may simultaneously exhibit the effects of inhibiting cancer cell proliferation, reducing cancer cell migration and inhibiting angiogenesis, and thus may be used as an excellent anticancer agent. These extracellular vesicles may be administered in combination with a conventional cancer chemotherapeutic agent or cancer immunotherapeutic agent.

Another aspect of the present invention is directed to a pharmaceutical composition for preventing or treating cancer containing: a Faecalibacterium sp. strain; and a pharmaceutically acceptable carrier or excipient. The strain may be alive or pasteurized or heat-killed.

The Faecalibacterium sp. strain or Faecalibacterium sp. strain-derived extracellular vesicles (EVs) of the present invention and the cancer chemotherapeutic agent or cancer immunotherapeutic agent may be administered simultaneously in a single dosage form, or may be administered simultaneously or sequentially in separate dosage forms.

In the present invention, a method for isolating extracellular vesicles is not limited. For example, these extracellular vesicles may be isolated from a culture of the Faecalibacterium sp. strain by centrifugation, ultra-high-speed centrifugation, filtration through a filter, gel filtration chromatography, free-flow electrophoresis, capillary electrophoresis, isolation using a polymer, or a combination thereof. Preferably, the extracellular vesicles may be isolated by centrifugation/ultracentrifugation. In this regard, centrifugation/ultracentrifugation may be performed sequentially at 100 to 300,000 g, preferably 150 to 150,000 g to remove cell debris, non-extracellular vesicle fractions, killed bacteria, and the like.

Differential centrifugation: The most preferred method for extracellular vesicles is differential centrifugation. This method consists of several steps, is preferably carried out at about 4° C., and includes at least the following three steps 1) to 3):

step 1) low-speed centrifugation to remove cells and cell debris;

step 2) high-speed spinning to remove large vesicles >100 nm; and

step 3) high-speed centrifugation to pellet extracellular vesicles.

Density gradient centrifugation: This approach combines ultracentrifugation with a sucrose density gradient. More specifically, density gradient centrifugation is used to separate extracellular vesicles from non-vesicular particles, such as proteins and protein/RNA aggregates. Thus, this method separates vesicles from particles of different densities.

Size exclusion chromatography: Size exclusion chromatography is used to separate macroparticles based on size, not molecular weight. This technique applies a column packed with porous polymer beads containing multiple pores and tunnels. Molecules pass through the beads depending on their diameter. It takes a longer time for molecules with small radii to migrate through pores of the column, while macromolecules elute earlier from the column. Size-exclusion chromatography allows precise separation of large and small molecules.

Filtration: Ultrafiltration membranes may also be used for isolation of exosomes. Depending on the size of microvesicles, this method allows the separation of exosomes from proteins and other macromolecules.

Polymer-based precipitation: Polymer-based precipitation technique usually includes mixing the biological fluid with polymer-containing precipitation solution, incubation at 4° C. and centrifugation at low speed. One of the most common polymers used for polymer-based precipitation is polyethylene glycol (PEG). The precipitation with this polymer has a number of advantages, including mild effects on isolated exosomes and usage of neutral pH.

Isolation by sieving: This technique isolates extracellular vesicles by sieving them from biological liquids via a membrane and performing filtration by pressure or electrophoresis.

The pharmaceutical composition of the present invention has an excellent effect on the prevention or treatment of cancer.

Specifically, the Faecalibacterium sp. strain or the extracellular vesicles derived from the Faecalibacterium sp. strain are uptaken into cancer cells, inhibit EMT activity, and activate the immune system, thereby inhibiting cancer cell invasion and metastasis.

Preferably, the Faecalibacterium sp. strain may be a Faecalibacterium prausnitzii EB-FPDK3 strain, an F. prausnitzii EB-FPDK9 strain, an F. prausnitzii EB-FPDK11 strain, or an F. prausnitzii EB-FPYYK1 strain. Preferably, the extracellular vesicles derived from the Faecalibacterium sp. strain may be extracellular vesicles derived from the Faecalibacterium prausnitzii EB-FPDK3 strain, the F. prausnitzii EB-FPDK9 strain, the F. prausnitzii EB-FPDK11 strain, or the F. prausnitzii EB-FPYYK1 strain. Hereinafter, these extracellular vesicles will be abbreviated as EB-FPDK3 EVs, EB-FPDK9 EVs, EB-FPDK11 EVs, and EB-FPYYK1 EVs, respectively. These EB-FPDK3 EVs, EB-FPDK9 EVs, EB-FPDK11 EVs, and EB-FPYYK1 EVs play an important role in activating innate and adaptive immune systems by regulating T cells. In addition, regulatory T cells (Treg), characterized by the expression of Foxp3, are known to suppress anticancer immunity, thereby impeding protective immune surveillance of tumors and hindering effective antitumor immune responses, but EB-FPDK3 EVs, EB-FPDK9 EVs, EB-FPDK11 EVs, and EB-FPYYK1 EVs are presumed to exhibit an anticancer effect by activating T helper cells, thereby activating cytotoxic T cells and suppressing Treg cells. Accordingly, the pharmaceutical composition of the present invention has excellent effects on the prevention, treatment, and suppression of metastasis of cancer.

As used herein, the term “cancer” is meant to include tumors, neoplasias, and malignant tissues or cells. Examples of the cancer include colorectal cancer, lung cancer, small cell lung cancer, gastric cancer, liver cancer, blood cancer, bone cancer, pancreatic cancer, skin cancer, head or neck cancer, skin or intraocular melanoma, uterine cancer, ovarian cancer, rectal cancer, perianal cancer, colon cancer, breast cancer, fallopian tube carcinoma, endometrial carcinoma, cervical cancer, vaginal cancer, vulvar carcinoma, Hodgkin's disease, esophageal cancer, small intestine cancer, endocrine adenocarcinoma, thyroid cancer, parathyroid cancer, adrenal cancer, soft tissue sarcoma, urethral cancer, penis cancer, prostate cancer, chronic or acute leukemia, lymphocytic lymphoma, bladder cancer, kidney cancer, ureteral cancer, renal cell carcinoma, renal pelvic carcinoma, CNS tumor, primary CNS lymphoma, spinal cord tumor, brainstem glioma, pituitary adenoma, or a combination of two or more of these cancers.

In the present invention, the Faecalibacterium sp. strain-derived extracellular vesicles (EVs) according to the present invention, which are contained as an active ingredient in the pharmaceutical composition, have a size of 20 to 300 nm.

The effective amount of the pharmaceutical composition according to the present invention may vary depending on the patient's age, sex, and body weight, and may generally be administered daily or every other day or administered 1 to 3 times, at a dose of 0.001 to 150 mg/kg body weight, preferably 0.01 to 100 mg/kg body weight.

The pharmaceutical composition according to the present invention may be used as a single anticancer agent. In addition, the pharmaceutical composition according to the present invention may be used simultaneously, separately or sequentially with radiotherapy, chemotherapy or immunotherapy, if necessary, depending on the circumstances. The combination therapy of the present invention is intended for use in at least one of reducing tumor size, reducing tumor growth, preventing metastasis, or preventing angiogenesis.

Specifically, the pharmaceutical composition of the present invention may be administered sequentially or simultaneously with conventional radiotherapy or anticancer therapeutic agents. In addition, it may be administered once or multiple times, and it is important to administer the pharmaceutical composition in the minimum amount that may exhibit the maximum effect without side effects, in consideration of all the above factors.

In some embodiments, the immunotherapeutic agent is an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors may be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STI-A1010.

Unlike conventional chemotherapy that directly kills cells, immune checkpoint inhibitors are attracting attention as next-generation anticancer agents that have few side effects, such as hair loss, anemia, and suppression of bone marrow function, which reduce the quality of life of cancer patients, compared to anticancer chemotherapy. However, immune checkpoint inhibitors are known to have very low response rates for some cancers (e.g., gastric cancer, colorectal cancer, ovarian cancer, pancreatic cancer, etc.) and cause severe immune-related adverse reactions such as enteritis, hepatitis, pneumonia, hypothyroidism, and pituitary glanditis. It has been reported that the side effects of using immune checkpoint inhibitors mostly appear as minor side effects, but are serious and fatal when they occur rarely in the nervous system or cardiac system. The pharmaceutical composition containing the Faecalibacterium sp. strain-derived extracellular vesicles according to the present invention may overcome the response rate limitations of immune checkpoint inhibitors, minimize side effects, and enhance anticancer efficacy.

The Faecalibacterium prausnitzii EB-FPDK3, F. prausnitzii EB-FPDK9, F. prausnitzii EB-FPDK11, and F. prausnitzii EB-FPYYK1 strains that are used in the present invention are mucin-degrading bacteria isolated from healthy Korean feces, which have ellipsoidal cells with a size of 0.5 to 1 μm, and are monococci or diplococci. These strains are anaerobic, non-motile, and Gram-negative, and do not form an endospore. The Faecalibacterium prausnitzii EB-FPDK3, F. prausnitzii EB-FPDK9, F. prausnitzii EB-FPDK11, and F. prausnitzii EB-FPYYK1 strains are capable of producing several mucolytic enzymes, and thus may use mucus as carbon and nitrogen sources. These strains may metabolize various carbon sources, including galactose, N-acetylglucosamine, and lactose, and produce, as main metabolites, short-chain fatty acids such as propionic acid and acetic acid.

The Faecalibacterium sp. strain in the pharmaceutical composition for preventing or treating cancer according to the present invention may be selected from among cells of the strain, a lysate of the cells, a culture of the strain, a culture medium obtained by removing cells from the culture of the strain, an extract from the cells of the strain, an extract from the culture of the strain, and an extract from the culture medium obtained by removing cells from the culture of the strain.

In the present invention, the Faecalibacterium sp. strain may be alive or pasteurized or heat-killed. The Faecalibacterium prausnitzii EB-FPDK3, F. prausnitzii EB-FPDK9, F. prausnitzii EB-FPDK11, and F. prausnitzii EB-FPYYK1 strains of the present invention may be recovered by a separation process such as centrifugation, and prepared as a probiotic by drying, for example, freeze-drying, for use. The killed bacterium may be a killed bacterium obtained by heat treatment or a bacterium inactivated by pasteurization. Pasteurization of the Faecalibacterium prausnitzii strain means heating the strain at a temperature of 50° C. to less than 100° C. for 10 minutes or more. For example, the strain may be pasteurized at 70° C. for 30 minutes.

As used herein, the term “killed bacteria” refers to bacteria sterilized by heating, pressurization, drug treatment, etc. As a method for producing killed bacteria, any method for killing lactic acid bacteria known in the art may be used without particular limitation. As an example, the killed bacteria of the present invention may be produced by a killing method including heat treatment or tyndalization. The heat treatment may be performed only on alive bacteria separated from a culture medium, or may be performed on the culture medium containing alive bacteria. Although the heat treatment temperature may be any temperature at which the properties of the cells are maintained and other general bacteria are sterilized, the heat treatment may be performed at a temperature of 80° C. to 150° C., preferably 80° C. to 110° C.

In one embodiment of the present invention, for use, the pharmaceutical composition containing the extracellular vesicles derived from the Faecalibacterium prausnitzii EB-FPDK3, F. prausnitzii EB-FPDK9, F. prausnitzii EB-FPDK11, or F. prausnitzii EB-FPYYK1 strain may be formulated into oral preparations such as powders, granules, tablets, capsules, suspensions, emulsions, syrups or aerosols, preparations for external use, suppositories, and sterile injectable solutions, according to the respective conventional methods, without being limited thereto.

In some embodiments, dosage forms of the pharmaceutical composition of the present invention include, but are not limited to, aqueous liquid dispersions, self-emulsifying dispersions, solid solutions, liposomal dispersions, aerosols, solid body forms, powders, immediate release formulations, controlled release formulations, fast melt formulations, tablets, capsules, pills, delayed release formulations, extended release formulations, pulsatile release formulations, multiparticulate formulations, and mixed immediate release and controlled release formulations.

For intravenous, intratumoral or intranasal administration of the pharmaceutical composition of the present invention, aqueous suspensions, isotonic saline solutions, or sterile, injectable solutions that contain pharmacologically compatible dispersing agents and/or wetting agents may be used. As an excipient, water, alcohols, polyols, glycerol, vegetable oils, etc., may be used.

The pharmaceutical composition of the present invention may be formulated as a product for enteral or oral administration. In addition, the pharmaceutical composition of the present invention may be productized by enteric coating using any known method so that it can pass through the stomach and then reach the small intestine in which the active ingredient extracellular vesicles (EVs) can be rapidly released into the intestines.

The pharmaceutical composition of the present invention may further contain pharmaceutically acceptable carriers and/or excipients, in addition to the active ingredient. In addition, the composition may be formulated with various additives, such as a binder, a disintegrant, a coating agent, and a lubricant, which are commonly used in the pharmaceutical industry.

Pharmaceutically acceptable carriers include, for example, carriers for oral administration or carriers for parenteral administration. Carriers for oral administration include lactose, starch, cellulose derivatives, magnesium stearate, stearic acid and the like. In addition, various drug delivery materials that are used for oral administration may be included. In addition, carriers for parenteral administration include water, suitable oil, saline, aqueous glucose, glycol, and the like. The pharmaceutical composition of the present invention may further contain a stabilizer and a preservative. Suitable stabilizers include antioxidants such as sodium bisulfite, sodium sulfite or ascorbic acid. Suitable preservatives include benzalkonium chloride, methyl- or propyl-paraben, and chlorobutanol. For pharmaceutically acceptable carriers and agents that may be contained in the pharmaceutical composition of the present invention, reference may be made to Remington's Pharmaceutical Sciences, 19^{th} ed., Mack Publishing Company, Easton, PA, 1995.

Excipients that may be used in the present invention include sugars such as sucrose, lactose, mannitol, or glucose; and starches such as corn starch, potato starch, rice starch, or partially pregelatinized starch. Binders that may be used in the present invention include polysaccharides such as dextrin, sodium alginate, carrageenan, guar gum, acacia, and agar; naturally occurring macromolecular substances such as tragacanth, gelatin, and gluten; cellulose derivatives such as hydroxypropylcellulose, methylcellulose, hydroxypropyl methyl cellulose, ethyl cellulose, hydroxypropyl ethyl cellulose, and sodium carboxymethyl cellulose; and polymers such as polyvinylpyrrolidone, polyvinyl alcohol, polyvinyl acetate, polyethylene glycol, polyacrylic acid, polymethacrylic acid, and vinyl acetate resin.

Disintegrants that may be used in the present invention include: cellulose derivatives such as carboxymethylcellulose, calcium carboxymethylcellulose, low-substituted hydroxypropylcellulose, and cellulose derivatives; and starches such as sodium carboxymethyl starch, hydroxypropyl starch, corn starch, potato starch, rice starch, and partially pregelatinized starch.

Examples of lubricants that may be used in the present invention include talc, stearic acid, calcium stearate, magnesium stearate, colloidal silica, hydrous silicon dioxide, and various types of waxes and hydrogenated oils.

Coating agents that may be used in the present invention include, but are not necessarily limited to, water-insoluble copolymers such a as dimethylaminoethyl methacrylate-methacrylic acid copolymer, polyvinylacetal diethylaminoacetate, an ethylacrylate-methacrylic acid copolymer, an ethylacrylate-methylmethacrylate-chlorotrimethylammonium ethylmethacrylate copolymer, and ethyl cellulose; enteric polymers such as a methacrylic acid-ethyl acrylate copolymer, hydroxypropyl methyl cellulose phthalate, hydroxypropyl methyl cellulose acetate succinate; and water-soluble polymers such as methyl cellulose, hydroxypropyl methyl cellulose, polyvinylpyrrolidone, and polyethylene glycol.

The dosage of the Faecalibacterium sp. strain-derived extracellular vesicles as an active ingredient in the pharmaceutical composition for preventing or treating cancer according to the present invention may be determined in consideration of various factors, including the type of disease, the patient's age, body weight, sex and medical condition, the severity of the condition, sensitivity to the drug, the duration of administration, the route of administration, the route of administration, excretion rate, and drugs used in combination with the composition, as well as other factors well known in the medical field. Thus, the dose regime can vary widely, but it is important to administer the pharmaceutical composition in the minimum amount that can exhibit the maximum effect without causing side effects, in view of all the above-described factors, and this amount can be easily determined using standard methods by a person skilled in the art.

Another aspect of the present invention is directed to a health functional food containing a Faecalibacterium sp. strain or extracellular vesicles (EVs) derived from the Faecalibacterium sp. strain. The Faecalibacterium sp. strain may be alive or pasteurized or heat-killed. The extracellular vesicles are preferably extracellular vesicles derived from Faecalibacterium prausnitzii EB-FPDK3, F. prausnitzii EB-FPDK9, F. prausnitzii EB-FPDK11, or F. prausnitzii EB-FPYYK1 strains. The health functional food of the present invention may be used to prevent or ameliorate cancer.

As used herein, the term “health functional food” is meant to include all forms, including neutraceutical foods, nutritional supplements, health foods, food additive, and feed.

These types of health functional food may be prepared in various forms according to conventional methods known in the art. General foods include, but are not limited to, beverages (including alcoholic beverages), fruits and their processed foods, fish, meat and their processed foods, bread and noodles, fruit juice, various drinks, cookies, taffy, dairy products, edible plant oils, margarine, vegetable proteins, retort food, frozen food, various sauces, etc., and these foods may be prepared by adding extracellular vesicles derived from Faecalibacterium prausnitzii EB-FPDK3, F. prausnitzii EB-FPDK9, F. prausnitzii EB-FPDK11, and F. prausnitzii EB-FPYYK1 strains thereto.

In addition, the health functional food of the present invention may further contain various nutrients, vitamins, electrolytes, flavoring agents, colorants, pectic acid or its salt, alginic acid or its salt, organic acids, protective colloidal thickeners, pH adjusting agents, stabilizers, preservatives, glycerin, alcohol, carbonizing agents, or the like.

Still another aspect of the present invention is directed to a veterinary composition for preventing or treating cancer containing: a Faecalibacterium sp. strain or extracellular vesicles (EVs) derived from the Faecalibacterium sp. strain; and a physiologically acceptable carrier or excipient. In this case, the animal is not particularly limited, and may refer to pets such as dogs, cats, guinea pigs, hamsters, rats, mice, ferrets, rabbits, and the like. The veterinary composition may be a veterinary drug or feed additive.

Yet another aspect of the invention is directed to a method of treating cancer in a subject. The method of the present invention includes a step of administering to a subject a therapeutically effective amount of the Faecalibacterium sp. strain or Faecalibacterium sp. strain-derived extracellular vesicles described herein.

Still yet another aspect of the present invention provides a novel Faecalibacterium sp. EB-FPDK3 strain (KCCM12619P), EB-FPDK9 strain (KCCM12620P), EB-FPDK11 strain (KCCM12621P), and EB-FPYYK1 strain (KCCM12622P). These strains were deposited with the Korean Collection for Type Cultures, the Korea Research Institute of Bioscience and Biotechnology, on Nov. 1, 2019.

Hereinafter, the present invention will be described in more detail with reference to examples. However, these examples are merely to illustrate the present invention, and the scope of the present invention is not limited to these examples.

### EXAMPLES

### Example 1: Isolation and Identification of Faecalibacterium prausnitzii EB-FPDK3, F. prausnitzii EB-FPDK9, F. prausnitzii EB-FPDK11, and F. prausnitzii EB-FPYYK1 strains

### 1.1. Isolation and Identification of Strains

In order to isolate Faecalibacterium sp. strains from the feces of a healthy Korean (female, 7 years old, BMI: 19.9), according to the method of Martin, the feces were cultured using YBHI medium [brain-heart infusion medium supplemented with 0.5% yeast extract, 0.1% D-cellobiose and 0.1% D-maltose] (Difco, Detroit, USA) in an anaerobic chamber under strict anoxic conditions (5% H₂, 5% CO₂ and 90% N₂), and then extremely oxygen sensitive (ECS) strains were selected and isolated (Martin et al., 2017). The type strain Faecalibacterium prausnitzii A2-165 was obtained from DSMZ (Deutsche Sammlung von Mikroorganismen und Zellkulturen) and used in experiments.

### 1.2. Microscopic Observation

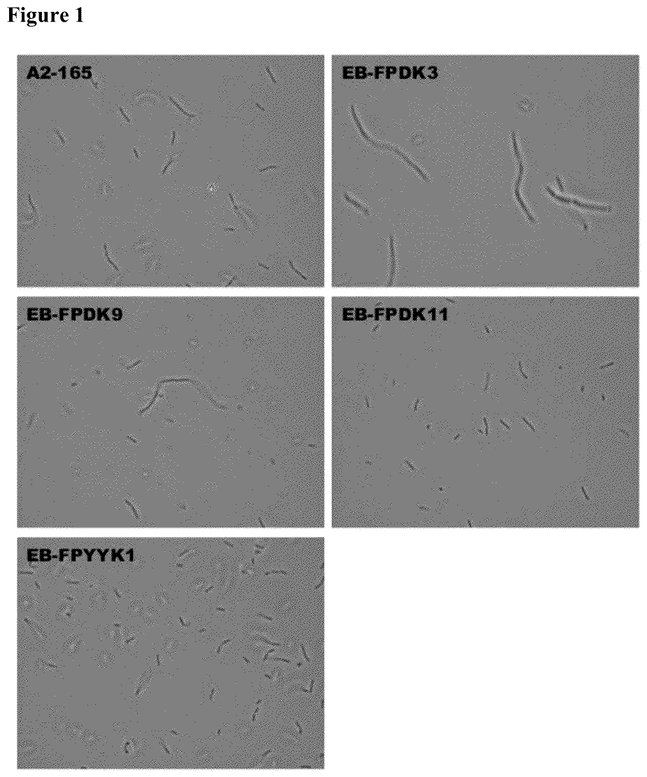

In order to confirm that the isolated strains would be Faecalibacterium sp. strains, the isolated strains were observed under a microscope, and the results are shown in FIG. 1. As shown in FIG. 1, as a result of observing the type strain Faecalibacterium prausnitzii A2-165 strain and the isolated Faecalibacterium prausnitzii EB-FPDK3, EB-FPDK9, EB-FPDK11 and EB-FPYYK1 strains of the present invention at 1,000× magnification, it was confirmed that these strains all had a straight or curved rod shape.

### 1.3. PCR Analysis

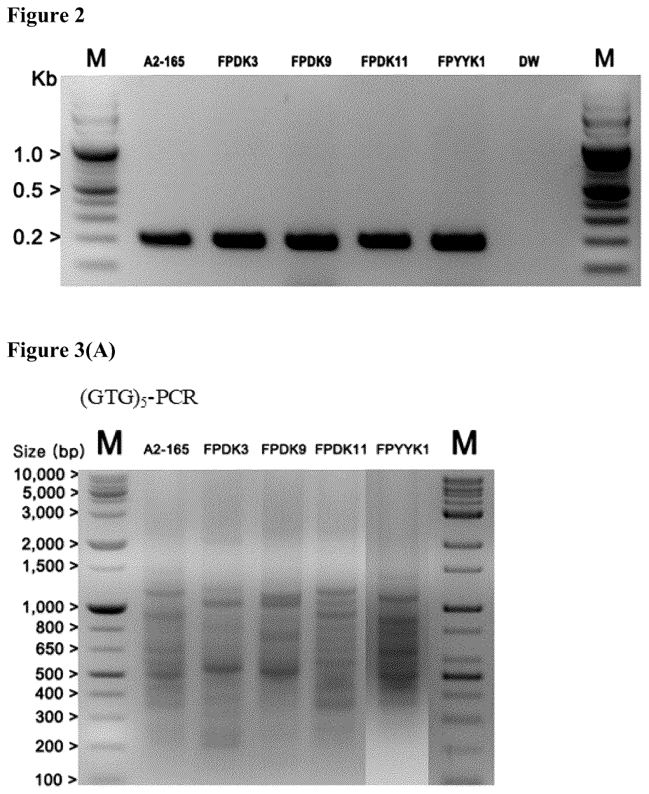

In order to confirm that the isolated strains would be Faecalibacterium sp. strains, the isolated strains were subjected to PCR analysis using FP-specific primers (SEQ ID NOS: 1 and 2), and the results are shown in FIG. 2. In FIG. 2, M represents a DNA size marker, lane 1 represents a positive control (A2-165), lanes 2 to 5 represent the isolated strains, and lane 6 represents a negative control (distilled water).

As shown in FIG. 2, it could be confirmed that the isolated Faecalibacterium prausnitzii EB-FPDK3, EB-FPDK9, EB-FPDK11 and EB-FPYYK1 strains of the present invention of the present invention showed the same band as that of the type strain the type strain Faecalibacterium prausnitzii A2-165.

TABLE 1

| Desig- | | | Amplicon | |
|---|---|---|---|---|
| nation | Direction | Sequence (5′→3′) | size | SEQ ID NO. |
| FP1 | Forward | ACT CAA CAA GGA AGT GA | 192 bp | SEQ ID NO: 1 |
| FP2 | Reverse | AAT TCC GCC TAC CTC TG | | SEQ ID NO: 2 |

### 1.4. Random Amplified Polymorphic DNA (RAPD) Analysis

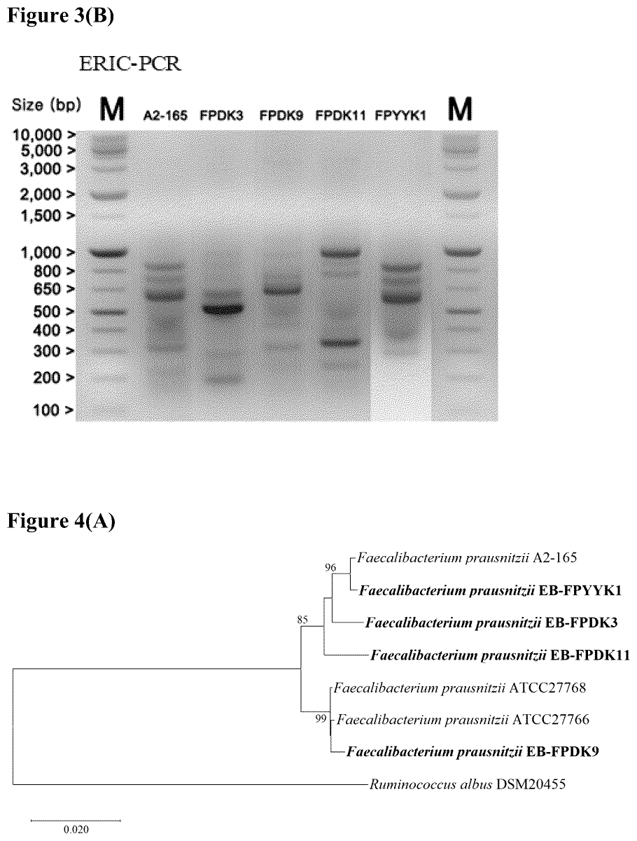

In order to verify the similarity of the Faecalibacterium prausnitzii EB-FPDK3, F. prausnitzii EB-FPDK9, F. prausnitzii EB-FPDK11, and F. prausnitzii EB-FPYYK1 strains isolated as described above to the previously reported type strain Faecalibacterium prausnitzii A2-165 of the same species, random amplified polymorphic DNA (RAPD) printing, which is a kind of molecular typing, was performed. To this end, the genomic DNA extracted from each strain was amplified using the universal primers shown in Table 2 below and were electrophoresed on 1% agarose gel for 1 hour and 30 minutes, and DNA fragmentation patterns were compared on a UV perforator. The results are shown in FIG. 3.

TABLE 2

| Desig- | | | |
|---|---|---|---|
| nation | Direction | Sequence (5′→3′) | SEQ ID NO. |
| ERIC-1 | Forward | ATG TAA GCT CCT GGG GAT TCA C | SEQ ID NO: 3 |
| ERIC-2 | Reverse | AAG TAA GTG ACT GGG GTG AGC G | SEQ ID NO: 4 |
| (GTG)5 | Forward/Reverse | GTG GTG GTG GTG GTG | SEQ ID NO: 5 |

As can be seen in FIG. 3, the Faecalibacterium prausnitzii EB-FPDK3, F. prausnitzii EB-FPDK9, F. prausnitzii EB-FPDK11, and F. prausnitzii EB-FPYYK1 strains of the present invention showed an RAPD band pattern different from that of the type strain Faecalibacterium prausnitzii A2-165. Since it is known that the RAPD band patterns of Faecalibacterium prausnitzii species are different from each other when the species are different, it was confirmed that the Faecalibacterium prausnitzii EB-FPDK3, F. prausnitzii EB-FPDK9, F. prausnitzii EB-FPDK11, and F. prausnitzii EB-FPYYK1 strains of the present invention belong to the same species as the type strain Faecalibacterium prausnitzii A2-165, but are different from the type strain.

1.5. Phylogenetic Tree Analysis Using Full-Length 16S rRNA Gene Sequence

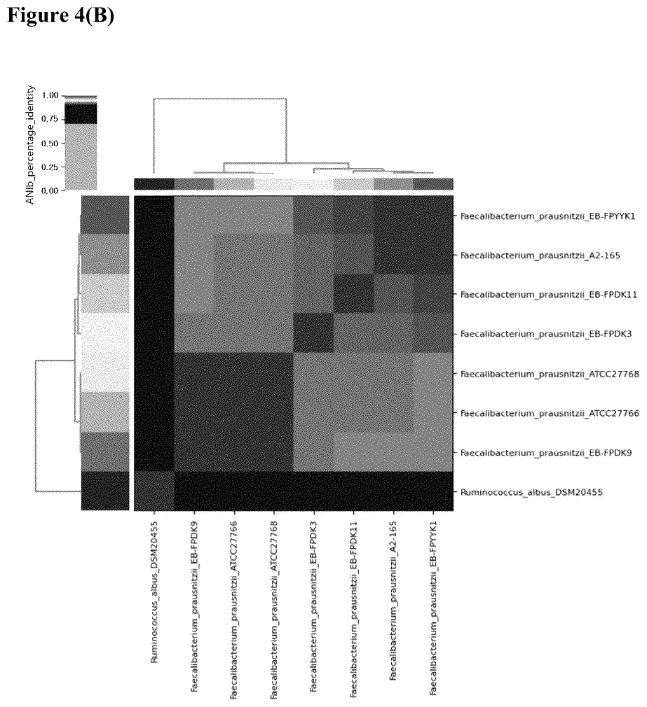

In order to analyze the full-length 16S rRNA gene sequences of the Faecalibacterium prausnitzii EB-FPDK3, F. prausnitzii EB-FPDK9, F. prausnitzii EB-FPDK11, and F. prausnitzii EB-FPYYK1 strains isolated as described above, the 16S rRNA genes were amplified using the 27F and 1541R primers shown in Table 3 below, and then sequenced using a 3730X1 DNA analyzer. A complete rRNA sequence database was created by collecting the 16S rRNA gene sequences of the following eight strains: the Faecalibacterium prausnitzii EB-FPDK3, F. prausnitzii EB-FPDK9, F. prausnitzii EB-FPDK11, and F. prausnitzii EB-FPYYK1 strains isolated as described above, previously published Faecalibacterium prausnitzii strains (A2-165, ATCC2766, and ATCC27768), and a Ruminococcus albus DSM20455 strain belonging to the Ruminococcaceae family. In addition, identity and query coverage were calculated using the Nucleotide-Nucleotide BLAST 2.9.0+ program, and the results are shown in Table 4 below. In addition, a phylogenetic tree was prepared using the 16S rRNA gene sequences of the eight strains. Phylogenetic analysis was performed using MEGA-X, and a phylogenetic tree was prepared through a neighbor-joining method using 1,000 bootstraps, and is shown in FIG. 4(A). Based on average nucleotide identity (ANI) values, evolutionary distances were evaluated using the pyani v0.2.7 program with “-m ANIb” setting. The complete or draft genome sequences of the Faecalibacterium prausnitzii A2-165 strain (RefSeq assembly accession: GCF_000162015.1), the Faecalibacterium prausnitzii_ATCC27766 strain (RefSeq assembly accession: GCF_003324115.1), the Faecalibacterium prausnitzii_ATCC27768 strain (RefSeq assembly accession: GCF_003324185.1), and the Ruminococcus albus DSM20455 strain (RefSeq assembly accession: GCF_000179635.2) were downloaded from the NCBI genome database (https://www.ncbi.nlm.nih.gov/genome/) and used. A phylogenetic tree was prepared using the 16S rRNA gene sequences of other strains of the same species and is shown in FIG. 4(B).

TABLE 3

| | | | | Amplicon |
|---|---|---|---|---|
| Designation | Direction | Sequence (5′→3′) | SEQ ID NO. | size |
| 27F | Forward | AGA GTT TGA TCM TGG CTC AG | SEQ ID NO: 6 | 1,472 bp |
| 1492R | Reverse | GGT TAC CTT GTT ACG ACT T | SEQ ID NO: 7 | |

TABLE 4

| | F. prausnitzii | | F. prausnitzii | | Ruminococcus albus | |
|---|---|---|---|---|---|---|
| | A2-165 | | ATCC27768 | | DSM20455 | |
| | | Query | | Query | | Query |
| Strain | Identity % | coverage % | Identity % | coverage % | Identity % | coverage % |
| EB-FPYYK1 | 99.735 | 100 | 97.95 | 100 | 86.868 | 100 |
| EB-FPDK3 | 98.676 | 100 | 98.082 | 100 | 86.623 | 100 |
| EB-FPDK9 | 97.95 | 100 | 99.603 | 100 | 86.588 | 100 |
| EB-FPDK11 | 98.613 | 100 | 97.82 | 100 | 86.82 | 100 |

As shown in FIGS. 4(A) and 4(B) and Table 4 above, it was confirmed that the 16S rRNA gene sequence of the Faecalibacterium prausnitzii EB-FPYYK1 strain shared 99.735% identity and 100% query coverage with that of the previously published Faecalibacterium prausnitzii A2-165 strain, 97.95% identity and 100% query coverage with that of the ATCC27768 strain, and 86.868% identity and 100% query coverage with that of the Ruminococcus albus DSM20455 strain belonging to the Ruminococcaceae family. It was confirmed that the Faecalibacterium prausnitzii EB-FPDK3 strain had 98.676% identity to the A2-165 strain, 98.082% identity to the F. prausnitzii ATCC27768 strain, and 86.623% identity to the R. albus DSM20455 strain. It was confirmed that the EB-FPDK9 strain had 97.95% identity to the A2-165 strain, 99.603% identity to the ATCC27768 strain, and 86.623% identity to the R. albus DSM20455 strain. It was confirmed that the EB-FPDK11 strain had 98.613% identity to the type strain F. prausnitzii A2-165, 97.82% identity to the F. prausnitzii ATCC27768 strain, and 86.82% identity to the DSM20455 strain. That is, as a result of analyzing the phylogenetic tree and the evolutionary relationships through 16S rRNA gene sequence analysis, it was confirmed that the Faecalibacterium prausnitzii EB-FPDK3, F. prausnitzii EB-FPDK9, F. prausnitzii EB-FPDK11, and F. prausnitzii EB-FPYYK1 strains genetically belong to Faecalibacterium prausnitzii species.

The Faecalibacterium prausnitzii EB-FPDK3, F. prausnitzii EB-FPDK9, F. prausnitzii EB-FPDK11, and F. prausnitzii EB-FPYYK1 strains of the present invention, isolated from human feces, were identified through the biochemical method (API) and molecular biological methods (16s rRNA sequencing, 16S rRNA BLAST analysis, and RAPD) using Faecalibacterium prausnitzii (A2-165) as a control. In addition, through the antibiotic resistance test described below, the isolated strains were found to be safe strains that can function as probiotics. Based on these results, the isolated Faecalibacterium prausnitzii strains were named Faecalibacterium prausnitzii EB-FPDK3 strain, F. prausnitzii EB-FPDK9 strain, F. prausnitzii EB-FPDK11 strain, and F. prausnitzii EB-FPYYK1 strain, and deposited with the Korea Research Institute of Bioscience and Biotechnology under accession numbers KCCM12619P, KCCM12620P, KCCM12621P, and KCCM12622P, respectively.

### Example 2: Analysis of Mycological Characteristics and Safety of Faecalibacterium prausnitzii EB-FPDK3, F. prausnitzii EB-FPDK9, F. prausnitzii EB-FPDK11, and F. prausnitzii EB-FPYYK1 strains

### 2.1. Analysis of Antimicrobial Susceptibility of Isolated Strains

In order to examine the antimicrobial susceptibility of the Faecalibacterium prausnitzii strains isolated as described above, the minimum inhibitory concentrations (MICs) of antibiotics for anaerobic bacteria (piperacillin-tazobactam (PTZ), ceftizoxime (CTZ), chloramphenicol (CHL), clindamycin (CLI), meropenem (MEM), moxifloxacin (MXF), metronidazole (MTZ), and ciprofloxacin (CIP)) against the isolated strains were determined by broth microdilution according to the guideline of Clinical & Laboratory Standard Institute (CLSI, 2017), and the results are shown in Table 5 below.

TABLE 5

| | MICa Breakpoints | | | QC | Test strains | | | | | | | | | |
|---|---|---|---|---|---|---|---|---|---|---|---|---|---|---|
| | (μg/mL) | | | ATCC | | | EB- | | EB- | | EB- | | EB- | |
| Antibiotics | S | I | R | 29741^{a} | A2-165 | | FPDK3 | | FPDK9 | | FPDK11 | | FPYYK1 | |
| PTZ | ≤32/4 | 64/4 | ≥128/4 | 8/4 | >256/4 | (R) | 32/4 | (S) | 32/4 | (S) | >256/4 | (R) | >256/4 | (R) |
| CTZ | ≤32 | 64 | ≥128 | 16 | 64 | (I) | 16 | (S) | 128 | (R) | 128 | (R) | 128 | (R) |
| CHL | ≤8 | 16 | ≥32 | 8 | 64 | (R) | 8 | (S) | 32 | (R) | 8 | (S) | 256 | (R) |
| CLI | ≤2 | 4 | ≥8 | 4 | ≤0.125 | (S) | ≤0.125 | (S) | ≤0.125 | (S) | ≤0.125 | (S) | ≤0.125 | (S) |
| MEM | ≤4 | 8 | ≥16 | 0.5 | >64 | (R) | >64 | (R) | >64 | (R) | >64 | (R) | >64 | (R) |
| MXF | ≤2 | 4 | ≥8 | 8 | 16 | (R) | >32 | (R) | 32 | (R) | 32 | (R) | >32 | (R) |
| MTZ | ≤8 | 16 | ≥32 | 2 | 4 | (S) | 1 | (S) | <0.25 | (S) | 0.5 | (S) | 2 | (S) |
| CIP | ≤1 | 2 | ≥4 | >32 | 32 | (R) | 32 | (R) | >32 | (R) | 16 | (R) | 32 | (R) |

PTZ: Piperacillin-tazobactam,
CTZ: ceftizoxime (3^{rd} gen),
CHL: chloramphenicol,
CLI: clindamycin,
MEM: meropenem,
MXF: moxifloxacin (4^{th} gen),
MTZ: metronidazole,
CIP: ciprofloxacin (2^{nd} gen),
^{a}MIC: minimal inhibitory concentration,
^{b}Bacteroides thetiotaomicron ATCC 29741

As can be seen in Table 5 above, the Faecalibacterium prausnitzii strains of the present invention showed different resistance patterns, and the strains all exhibited susceptibility to metronidazole and all resistance to moxifloxacin and ciprofloxacin which are fluoroquinolone-based antibiotics. It is considered that the resistance of the strains to the fluoroquinolone-based antibiotics is intrinsic resistance that exists in the same Faecalibacterium prausnitzii. Additionally, based on the antibiotic resistance gene database MEGARes (https://megares.meglab.org/), the genes encoding the antibiotic resistance genes were examined in the whole genomes of the Faecalibacterium prausnitzii EB-FPDK3, EB-FPDK9, EB-FPDK11, and EB-FPYYK1 strains of the present invention, and the results are shown in Table 6 below.

TABLE 6

| Genome | MEGAResID | Ident | Align | Coverage | Class | Mechanism | Group |
|---|---|---|---|---|---|---|---|
| A2-165 | MEG_988 | 99.755 | 816 | 100 | Aminoglycosides | Aminoglycoside O- | ANT6 |
| | | | | | | nucleotidyltransferases | |
| EB-FPYYK1 | MEG_988 | 99.885 | 867 | 100 | Aminoglycosides | Aminoglycoside O- | ANT6 |
| | | | | | | nucleotidyltransferases | |
| EB-FPDK3 | MEG_2793 | 100 | 738 | 100 | MLS | 23S rRNA | ERMB |
| | | | | | | methyltransferases | |
| EB-FPDK9 | MEG_2793 | 100 | 738 | 100 | MLS | 23S rRNA | ERMB |
| | | | | | | methyltransferases | |
| EB-FPDK11 | MEG_7216 | 99.844 | 1920 | 100 | Tetracyclines | Tetracycline | TETW |
| | | | | | | resistance ribosomal | |
| | | | | | | protection proteins | |

As can be seen in Table 6 above, aminoglycoside O-nucleotidyltransferase gene was detected in the type strain Faecalibacterium prausnitzii. In addition, Aminoglycoside O-nucleotidyltransferase, 23S rRNA methyltransferase, 23S rRNA methyltransferase, and tetracycline resistance ribosomal protection protein genes were detected in the Faecalibacterium prausnitzii EB-FPYYK1, EB-FPDK3, EB-FPDK9, and EB-FPDK11 strains of the present invention, respectively. It was confirmed that the antibiotic resistance genes detected through the MEGARes database were also detected through Resfinder (https://cge.cbs.dtu.dk/services/ResFinder/), a bioinformatics-based antibiotic resistance gene detection program. From these results, it was confirmed that the antibiotic resistance genes did not appear equally in the Faecalibacterium prausnitzii strains, but appeared differently for each strain.

As the antibiotic resistance genes were detected in the Faecalibacterium prausnitzii strains, additional examination was performed using PlasmidFinder (https://cge.cbs.dtu.dk/services/PlasmidFinder/) and Mobile Element Finder (cge.cbs.dtu.dk/services/MobileElementFinder) programs.

As a result of examining the presence or absence of plasmids on the whole genomes of the Faecalibacterium prausnitzii EB-FPYYK1, EB-FPDK3, EB-FPDK9 and EB-FPDK11 strains using the PlasmidFinder program, it was confirmed that no plasmid was detected. In addition, as a result of examining the presence or absence of mobile genetic elements on the whole genomes of the Faecalibacterium prausnitzii EB-FPYYK1, EB-FPDK3, EB-FPDK9 and EB-FPDK11 strains through the Mobile Element Finder program, it was confirmed that no transposon was detected. From these results, it was confirmed that the antibiotic resistance genes detected were not transmitted to other strains. This suggests that the Faecalibacterium prausnitzii EB-FPDK3, F. prausnitzii EB-FPDK9, F. prausnitzii EB-FPDK11 and F. prausnitzii EB-FPYYK1 strains according to the present invention are safe strains.

### 2.3. Analysis of Hemolytic Activity and Virulence Factors of Isolated Strains

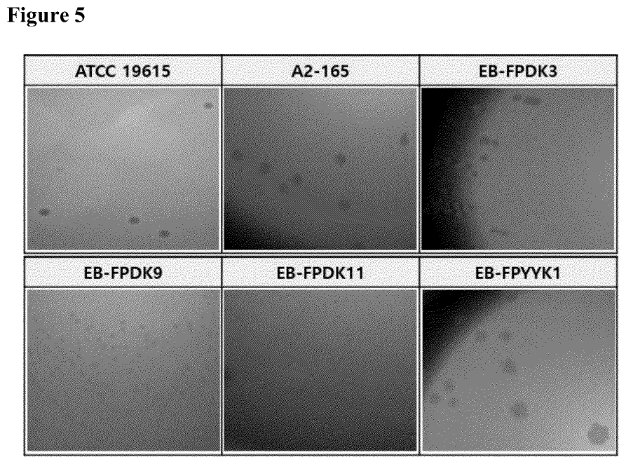

In order to verify the safety of the Faecalibacterium prausnitzii EB-FPDK3, F. prausnitzii EB-FPDK9, F. prausnitzii EB-FPDK11 and F. prausnitzii EB-FPYYK1 strains isolated as described above, whether the strains would have hemolytic activity was evaluated. To this end, each strain was cultured using a blood agar medium prepared by adding 5% w/v defibrinated sheep blood to tryptic soy agar (17.0 g/L pancreatic digest of casein, 3.0 g/L pancreatic digest of soybean, 2.5 g/L dextrose, 5.0 g/L sodium chloride, 2.5 g/L potassium phosphate, and 15 g/L agar). The results of the culture are shown in FIG. 5. As can be seen in FIG. 5, β-hemolysis (a fully transparent part around a colony) associated with pathogenicity was not observed in the Faecalibacterium prausnitzii EB-FPDK3, F. prausnitzii EB-FPDK9, F. prausnitzii EB-FPDK11 and F. prausnitzii EB-FPYYK1 strains of the present invention.

Based on the pathogenic bacteria database VFDB (reference database for bacterial virulence factors, http://www.mgc.ac.cn/VFs), genes encoding virulence factors were analyzed in the whole genomes of the Faecalibacterium prausnitzii EB-FPDK3, F. prausnitzii EB-FPDK9, F. prausnitzii EB-FPDK11 and F. prausnitzii EB-FPYYK1 strains of the present invention. The Virulence Factor Database (VFDB) is a comprehensive online resource for screening information on the virulence factors of bacterial pathogens, and provides in-depth coverage major virulence factors of the best characterized bacterial pathogens. The VFDB database used in analysis included 3,688 and 32,772 sequence data involved in virulence. Analysis was performed under the conditions of protein identity of at least 80%, coverage of at least 80%, and alignment length of at least 50 bp. As a result of the analysis, no virulence factors were detected in the Faecalibacterium prausnitzii EB-FPDK3, EB-FPDK9, EB-FPDK11 and EB-FPYYK1 strains of the present invention. Additionally, virulence factor genes were examined based on VirulenceFinder (https://cge.cbs.dtu.dk/services/VirulenceFinder/). The VirulenceFinder is a database of genome sequences of four well-known pathogens: E. coli, Enterococcus, Listeria, and Staphylococcus aureus. As a result, genes related to E. coli shiga toxin gene, S. aureus exoenzyme genes and host immune alteration or evasion genes and toxin genes were not detected in the whole genomes of the Faecalibacterium prausnitzii EB-FPDK3, EB-FPDK9, EB-FPDK11 and EB-FPYYK1 strains of the present invention. Overall, it can be seen that the Faecalibacterium prausnitzii EB-FPDK3, EB-FPDK9, EB-FPDK11 and EB-FPYYK1 strains of the present invention are harmless to the human body.

2.4. Evaluation of the Ability of Faecalibacterium prausnitzii Strains to Produce Short-Chain Fatty Acids (SCFAs)

Short-chain fatty acids (SCFAs), such as butyrate, acetate, and propionate, are metabolites produced by gut bacteria and play an important role in host energy metabolism. They are signaling mediators acting on G protein-coupled receptors (GPR41 and GPR43) and are involved in energy balance.

Short-chain fatty acids (SCFAs) decrease intestinal motility and increase intestinal transit rate, through GPR41 in enteroendocrine cells. Thereby, SCFAs induce PYY (peptide YY) secretion to reduce energy intake and prevent obesity. In addition, GPR43 by short-chain fatty acids induces GPL-1 (glucagon-like peptide 1) to increase insulin sensitivity, thereby increasing satiety, and the activity of GPR43 inhibits insulin signaling in adipose tissue to prevent fat accumulation. Short-chain fatty acids (SCFAs) can enhance glucose metabolism and activate intestinal gluconeogenesis (IGN), which can reduce food intake through the gut-brain neural circuit.

In order to identify changes in functional metabolites in the Faecalibacterium prausnitzii strains, the content of short-chain fatty acids (butyrate and acetate) contained in a culture of each strain was analyzed by gas chromatography (GC) after culturing in a test tube. To this end, the culture was centrifuged at 12,000 xg for 5 minutes, and the supernatant was collected, filtered through a 0.2 μm syringe filter, and then used for analysis. Analysis was performed using gas chromatography (Agilent 7890N) equipped with a FFAP column (30 m×0.320 mm, 0.25 μm phase) under the conditions were shown in Table 7 below, and the results of the analysis are shown in Table 8 below.

TABLE 7

| Flow | H₂: 40 mL/min, Air: 350 mL/min | |
|---|---|---|
| Injector temp. | 240° | C. |
| Detector temp. | 250° | C. |
| Oven temp. | 40° C. (hold for 2 min)→ | |
| | 65° C./10 min (hold for 2 min)→ | |
| | 240° C./10 min (hold for 5 min) | |
| Injection vol. | 2 | μL |

| | |
|---|---|
| Split ratio | 20:1 |

TABLE 8

| | | Short-chain fatty acid | | |
|---|---|---|---|---|
| | | production/ consumption | | |
| | | (μg/mL) | | |
| | Butyrate | | Acetate | |
| Strain | production | P value* | consumption | P value* |
| A2-165 | 508.75 ± 64.90 | — | 198.25 ± 13.47 | — |
| EB-FPDK3 | 326.44 ± 13.84 | <0.0001 | 246.10 ± 17.80 | <0.0001 |
| EB-FPDK9 | 358.83 ± 24.28 | <0.0001 | 160.89 ± 14.84 | <0.0001 |
| EB-FPDK11 | 354.22 ± 8.83 | <0.0001 | 154.42 ± 21.75 | <0.0001 |
| EB-FPYYK1 | 587.74 ± 17.46 | 0.0003 | 197.67 ± 7.25 | >0.9999 |

*P < 0.05 significantly different compared to A2-165 (type strain).

As can be seen from the results in Table 8 above, the Faecalibacterium prausnitzii EB-FPDK strains of the present invention exhibited different short-chain fatty acid production/consumption abilities. Specifically, it was confirmed that the amount of acetate consumed and the amount of butyrate produced were similar between the type strain Faecalibacterium prausnitzii A2-165 and the Faecalibacterium prausnitzii EB-FPYYK1, EB-FPDK9 and EB-FPDK11, and that the amount of butyrate produced by the EB-FPDK3 strain was the lowest among the five strains, but the amount of acetate consumed by the EB-FPDK3 strain was the highest. Faecalibacterium prausnitzii is known as representative butyric acid-producing bacteria, and butyrate is synthesized from acetate in their metabolic pathway, and thus acetate consumption occurs in the metabolic pathway.

### Example 3: Isolation of Extracellular Vesicles from Faecalibacterium prausnitzii Strains

To obtain extracellular vesicles (EVs) from the Faecalibacterium prausnitzii EB-FPDK3, F. prausnitzii EB-FPDK9, F. prausnitzii EB-FPDK11, and F. prausnitzii EB-FPYYK1 strains, a culture of each of the Faecalibacterium prausnitzii EB-FPDK3, F. prausnitzii EB-FPDK9, F. prausnitzii EB-FPDK11 and F. prausnitzii EB-FPYYK1 strains was subjected to high-speed centrifugation at 10,000xg at 4° C. for 20 minutes, and the supernatants were collected and filtered through a 0.45-μm filter and a 0.22-μm filter. Each of the filtered supernatants was subjected to high-speed centrifugation at 150,000xg at 4° C. for 2 hours to obtain pellets, which were then dissolved in sterile phosphate buffered saline (PBS) and used for protein quantification and then efficacy testing.

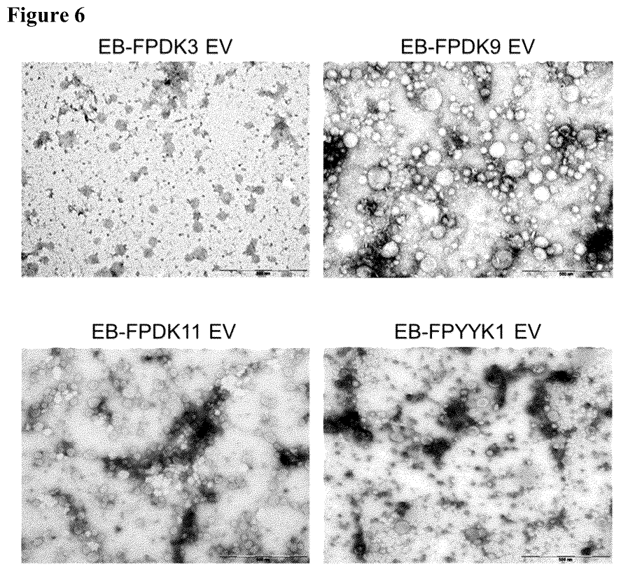

The isolated extracellular vesicles were observed under an electron microscope (Zeiss, Germany) (150,000× magnification), and the results are shown in FIG. 6. As shown in FIG. 6, it can be seen that the extracellular vesicles from the Faecalibacterium prausnitzii EB-FPDK3, F. prausnitzii EB-FPDK9, F. prausnitzii EB-FPDK11, and F. prausnitzii EB-FPYYK1 strains of the present invention are spherical in shape and have a size ranging from about 20 to 300 nm (scale bar: 500 nm).

### Example 4: Anticancer Effect of Co-Administration of Cancer Immunotherapeutic Agent aPD-1 and Alive Faecalibacterium prausnitzii EB-FPDK3, EB-FPDK9, EB-FPDK11, or EB-FPYYK1 Strains

### 4.1. Strain Samples

Alive Faecalibacterium prausnitzii A2-165 type strain (control), Faecalibacterium prausnitzii EB-FPDK3 strain (KCCM12619P), EB-FPDK9 strain (KCCM12620P), EB-FPDK11 strain (KCCM12621P), or EB-FPYYK1 strain (KCCM12622P) used in this experiment were prepared at a concentration of 1×10⁸ CFU/150 μl PBS (25% glycerol and 0.05% cysteine/PBS).

### 4.2. Animal Experiments

Animal experiments were conducted in compliance with the Animal Use and Care Protocol of the Institutional Animal Care and Use Committee (IACUC). For cancer induction, 8-week-old female C57BL/6 mice were purchased and acclimated for 1 week, Then, the mice were bred for 12 weeks. During breeding, the animals were kept at a constant temperature of 22° C. and a relative humidity of 40 to 60% with 12-hr light/12-hr dark cycles.

To prepare a syngeneic tumor animal model, mouse-derived melanoma cells (B16-F10) were used.

The syngeneic model is a technique in which a mouse cell line grown in vitro is transplanted into and grown in an actual mouse and grown, and the identical host and cell line strain means that tumor rejection does not occur.

After 1 week of acclimation, the mice were pretreated with the antibiotics shown in Table 9 below for 1 week.

TABLE 9

| Antibiotic | Concentration | |
|---|---|---|
| Ampicillin (Sigma-A0166) | 1 | g/L |
| Vancomycin (Sigma SBR00001) | 0.5 | g/L |
| Metronidazole (Sigma M1547) | 1 | g/L |
| Neomycin (Sigma N6386) | 1 | g/L |
| Amphotericin B (Sigma PHR1662) | 0.1 | g/L |

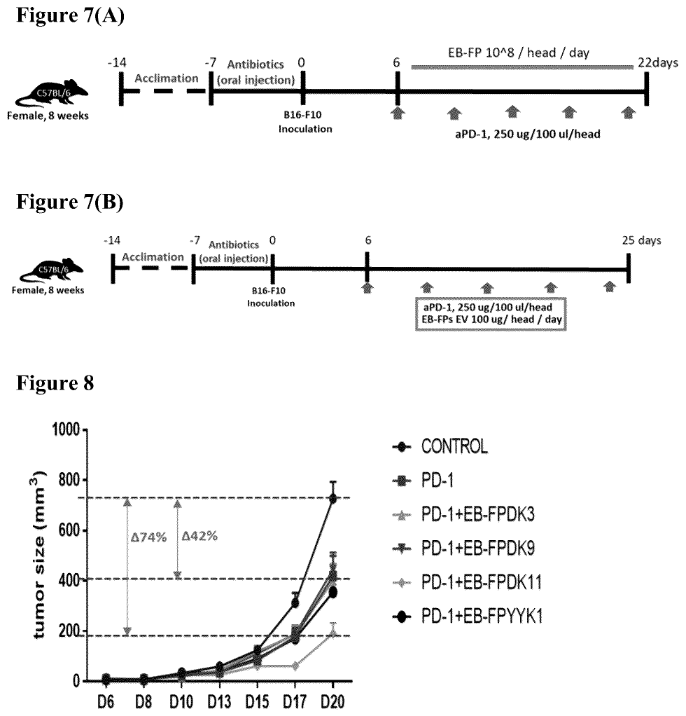

Subsequently, 2×10⁴ B16-F10 cells were subcutaneously (SC) injected into the thigh of each mouse together with 100 μl of Matrigel. The animal experiment scheme in this embodiment is shown in FIG. 7a.

### 4.3. Sample Administration and Experimental Group Setup

Each drug shown in Table 10 below was orally administered to mice every day for 2 weeks. As a positive control, anti-PD1 antibody was orally administered to each mouse at a concentration of 250 μg/100 μl/head.

Cancer cells began to appear on day 6, and from this time point, each of the Faecalibacterium prausnitzii EB-FPDK3, F. prausnitzii EB-FPDK9, F. prausnitzii EB-FPDK11 and F. prausnitzii EB-FPYYK1 strains was orally administered daily at 10⁸ CFU. From day 6, 250 μg of aPD-1 antibody was intraperitoneally injected every 4 days. In this case, as the aPD-1 antibody, InVivoMab anti-mouse PD-1 (RMP1-14) (catalog #BE0146, BioXCell) was used, which was diluted with InVivoPure pH 7.0 dilution buffer (catalog #IP0070) at a concentration of 250 μg/100 μl.

During tumor growth, the mice were weighed twice a week and monitored daily, and from day 6 when the cancer cells appeared, the tumor size was monitored every other day using a computerized caliper (see FIG. 8).

Tumor size was calculated according to the following equation by measuring the two diameters (major and minor diameters) of each tumor.

$$Tumor size ( {mm}^{3} ) = [ major diameter \times minor {diameter}^{2} ] / 2 \qquad [ Equation 1 ]$$

TABLE 10

| Group | Experimental group | Drug administered |
|---|---|---|
| I | Control group | PBS administered |
| II | Positive control group (anti-PD1- | 250 μg/100 μL/head |
| | administered group) | |
| III | Anti-PD1 + F. prausnitzii EB- | alive EB-FPDK3 strains (1 × 10⁸ CFU) + |
| | FPDK3-administered group | anti-PD1 (250 μg/100 μL/head) |
| IV | Anti-PD1 + F. prausnitzii EB- | alive EB-FPDK9 strains (1 × 10⁸ CFU) + |
| | FPDK9-administered group | anti-PD1 (250 μg/100 μL/head) |
| V | Anti-PD1 + F. prausnitzii EB- | alive EB-FPDK11 strains (1 × 10⁸ CFU) + |
| | FPDK11-administered group | anti-PD1 (250 μg/100 μL/head) |
| VI | Anti-PD1 + F. prausnitzii EB- | alive EB-FPYYK1 strains (1 × 10⁸ CFU) + |
| | FPYYK1-administered group | anti-PD1 (250 μg/100 μL/head) |

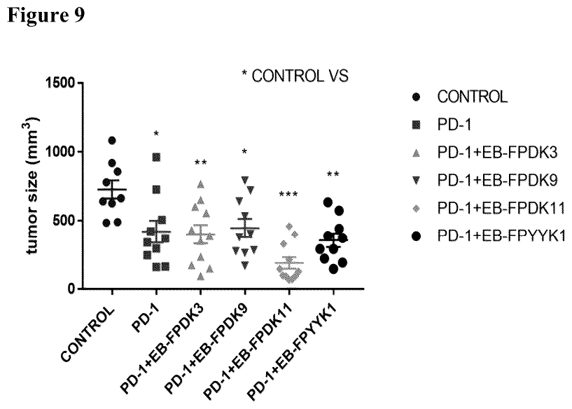

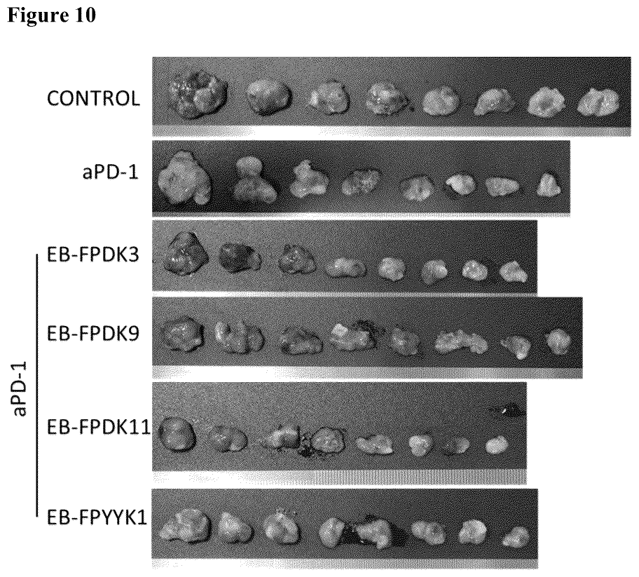

Referring to FIGS. 8 to 10, it was confirmed that the tumor size became significantly smaller in the group to which the alive Faecalibacterium prausnitzii EB-FPDK3, EB-FPDK9, EB-FPDK11 or EB-FPYYK1 strains were orally administered than in the control group into which the B16-F10 cells were syngeneically transplanted. It was confirmed that, compared to the tumor size in the control group (726.8±66.7 mm³), the tumor size in the aPD-1-administered group was 419.5±80.43 mm³ (42% decrease) (p<0.05). It was shown that the tumor sizes in the groups to which aPD-1 and each of EB-FPDK3, EB-FPDK9, EB-FPDK11 and EB-FPYYK1 were co-administered were 400.6±67.45 mm³ (P<0.01), 455.7±67.67 mm³ (P<0.05), 190.7±42.5 mm³ (P<0.001), and 355.6±50.47 mm³ (P<0.01), respectively, which significantly decreased. In particular, compared to the normal control group, the group to which aPD-1 and EB-FPDK11 were co-administered showed the best anticancer effect of reducing the tumor size by about 74% (p<0.05).

### Example 5: Anticancer Effect of Co-Administration of Cancer Immunotherapeutic Agent aPD-1 and Extracellular Vesicles (EVs) Derived from Faecalibacterium prausnitzii EB-FPDK3, EB-FPDK9, EB-FPDK11 or EB-FPYYK1 in Syngeneic Melanoma Mouse Animal Model

Referring to FIG. 7a, from day 6 when cancer cells began to appear, 250 μg of aPD-1 antibody and 100 μg of the extracellular vesicles (EVs) derived from the Faecalibacterium prausnitzii EB-FPDK3, F. prausnitzii EB-FPDK9, F. prausnitzii EB-FPDK11 and F. prausnitzii EB-FPYYK1 strains were intraperitoneally injected every 4 days. In this case, as the aPD-1 antibody, InVivoMab anti-mouse PD-1 (RMP1-14) (catalog #BE0146, BioXCell) was used, which was diluted with InVivoPure pH 7.0 dilution buffer (catalog #IP0070) at a concentration of 250 μg/100 μl.

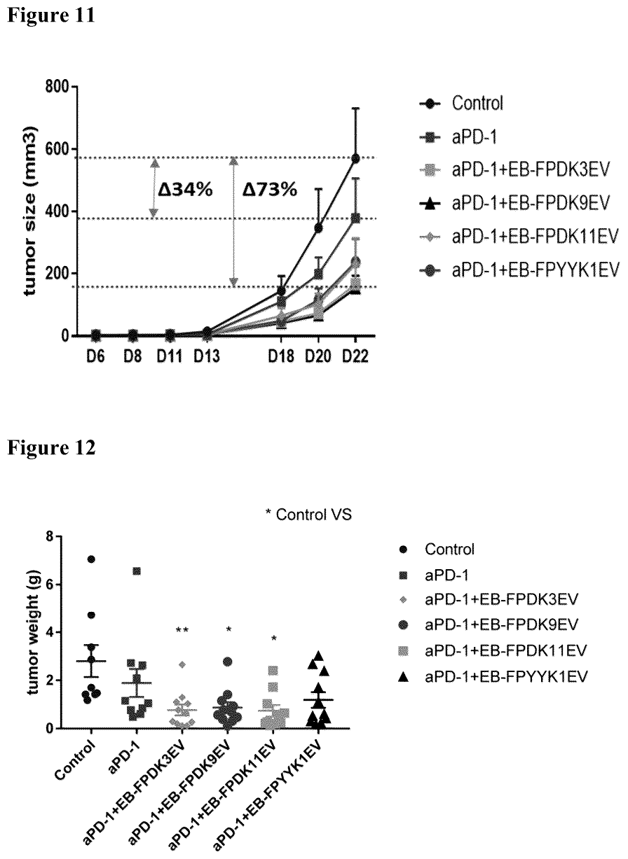

The tumor size was monitored once every two days from the time the tumor started to appear, and the tumor size was calculated according to Equation 1 above, and the results are shown graphically in FIG. 11. The extracellular vesicles were tested for their efficacy in the mouse tumor model alone or in the presence or absence of anti-PD1.

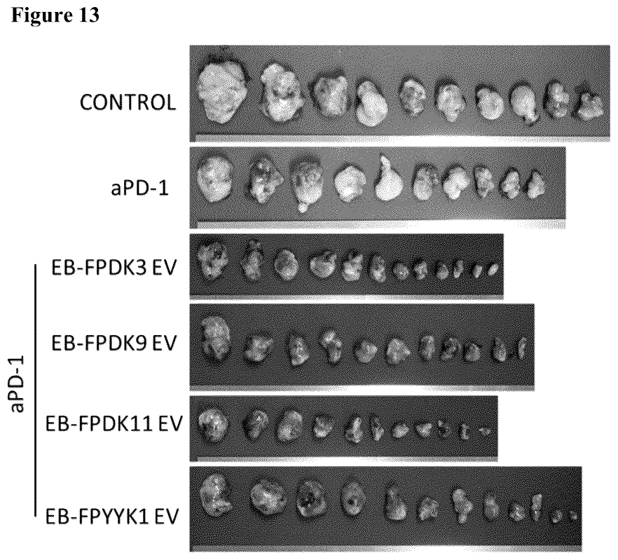

Referring to FIGS. 11 and 13, it was confirmed that the tumor size became significantly smaller in the groups, to which the extracellular vesicles (EVs) derived from each of the Faecalibacterium prausnitzii EB-FPDK3, F. prausnitzii EB-FPDK9, F. prausnitzii EB-FPDK11 and F. prausnitzii EB-FPYYK1 strains were orally administered, than in the control group into which the B16-F10 cells were syngeneically transplanted. It was confirmed that, compared to the tumor size in the control group (570.4±160.7 mm³), the tumor size in the aPD-1-administered group was 378.1±128 (about 34% decrease). It was shown that the tumor sizes in the groups to which aPD-1 and EB-FPDK3 EVs, aPD-1 and EB-FPDK9 EVs, aPD-1 and EB-FPDK11 EVs, and aPD-1 and EB-FPYYK1 EVs were co-administered were 165.6±57.54 mm³, 152.7±40.44 mm³, 230.2±83.91 mm³, and 237.1±73.95 mm³, respectively, which significantly decreased. In particular, the group to which aPD-1 and EB-FPDK9 EV were co-administered showed the best anticancer effect of reducing the tumor size by about 73% compared to the normal control group.

Referring to FIG. 12, as a result of measuring the weight of the tumor removed from each sacrificed mouse, it was confirmed that the average tumor weight of the aPD-1 group was 1.89±0.5806 g, which was not significantly different from that of the normal control group (2.8±0.6622 g). However, it was confirmed that the average tumor weight of the group to which PD-1 and each of EB-FPDK3 EVs, EB-FPDK9 EVs and EB-FPDK11 EVs significantly decreased compared to that of the normal control group. It was confirmed that the tumor weights of the groups to which PD-1 and EB-FPD EVs were co-administered were 0.7696±0.2281 g (P<0.01) in EB-FPDK3 EVs, 0.8688±0.2224 g (P<0.05) in EB-FPDK9 EVs, and 0.7409±0.2423 g (P<0.05) in EB-FPDK11 EVs, which significantly decreased compared to the tumor weight of the normal control group (2.8±0.6622 g).

As described above, the present inventors evaluated the cancer cell growth and metastasis inhibitory effects of the Faecalibacterium sp. strain-derived extracellular vesicles (EVs) and the immune checkpoint inhibitor anti-PD1 in vivo, and as a result, found that, when the Faecalibacterium sp. strain-derived extracellular vesicles (EVs) and the immune checkpoint inhibitor anti-PD1 were injected into the syngeneic tumor mice, the tumor size and weight effectively decreased (see FIGS. 11 and 12). Therefore, the pharmaceutical composition of the present invention has a significantly superior tumor growth inhibitory effect compared to anti-PD1 alone, and thus may be useful as a pharmaceutical composition for preventing or treating cancer.

### Example 6: Wound Healing Assay Using HT29 Cells and B16-F10

In order to examine the anti-cancer activity of the Faecalibacterium prausnitzii strain-derived extracellular vesicles of the present invention, a wound healing activity test was performed.

HT29 human colorectal cancer cells were cultured in McCoy's medium containing 10% FBS and 1% gentamicin at 37° C. under 5% CO₂. HT29 colorectal cancer cells were seeded in a 6-well plate for cell culture and cultured confluently. Thereafter, the 6-well plate was uniformly scratched using a pipette tip. Next, the cells were treated with 1 or 10 μg/ml of each of EB-FPDK3 EVs, EB-FPDK9 EVs, EB-FPDK11 EVs, and EB-FPYYK1 EVs for 24 hours and observed under a microscope. The cell area was calculated using the Image J program.

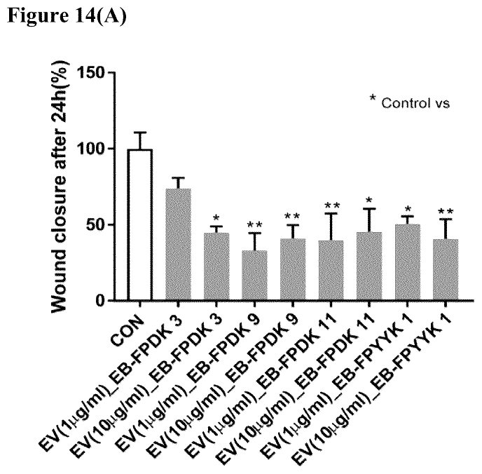

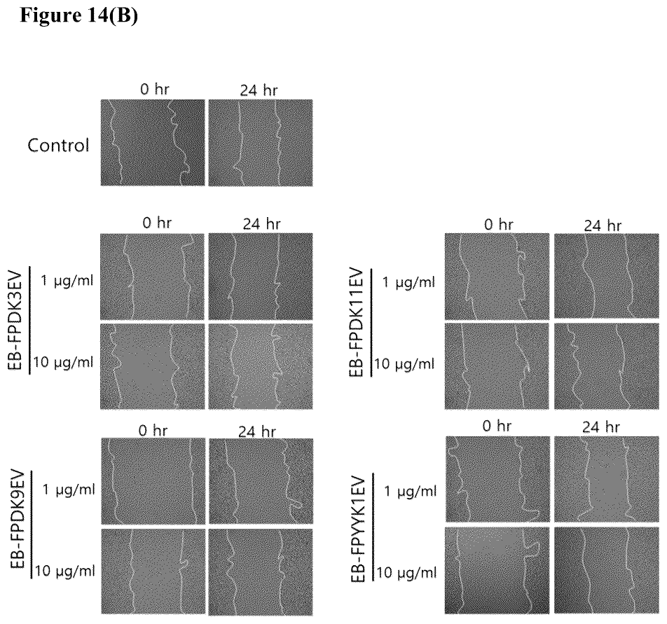

Referring to FIGS. 14a and 14b, as a result of the wound healing assay performed using HT29 cells, it was observed that the extents of metastasis in the groups treated with 1 μg/ml and 10 μg/ml of EB-FPDK3 EVs decreased by 25.97% and 55.25%, respectively, compared to that in the normal control group. In addition, the extent of metastasis significantly decreased in the group treated with 10 μg/ml of EB-FPDK3 EVs (P<0.05). In addition, it was observed that the extents of metastasis in the groups treated with 1 μg/ml and 10 μg/ml of EB-FPDK9 EVs decreased by 66.91% and 58.95%, respectively, compared to that in the normal control group, and significantly decreased at the two concentrations (P<0.05). It was observed that the extents of metastasis in the groups treated with 1 μg/ml and 10 μg/ml of EB-FPDK11 EVs decreased by 54.48% and 49.45%, respectively, compared to that in the normal control group, and significantly decreased at the two concentrations (P<0.05 and P<0.1, respectively). Finally, it was observed that the extents of metastasis in the groups treated with 1 μg/ml and 10 μg/ml of EB-FPYYK1 EVs decreased by 49.45% and 59.4%, respectively, compared to that in the normal control group, and significantly decreased at the two concentrations (P<0.1 and P<0.05, respectively).

### Example 7: Anticancer Effect of Administration of Faecalibacterium prausnitzii EB-FPDK9 Strain or Faecalibacterium prausnitzii EB-FPDK9 Strain-Derived Extracellular Vesicles (EB-FPDK9EVs) in Syngeneic Melanoma Mouse Animal Model

### 7.1 Experimental Method

5-week-old C57BL/6 female mice were purchased and acclimated for 1 week. Then, 5×10⁵ B16F10 melanoma cells were injected subcutaneously into the mice, thus preparing syngeneic mice. 250 μl of a mixture of 15 mg/ml of antibiotic ampicillin, 15 mg/ml of neomycin, 10 mg/ml of metronidazole, and 7.5 mg/ml of vancomycin was orally administered to all the mice once a day for 2 days.

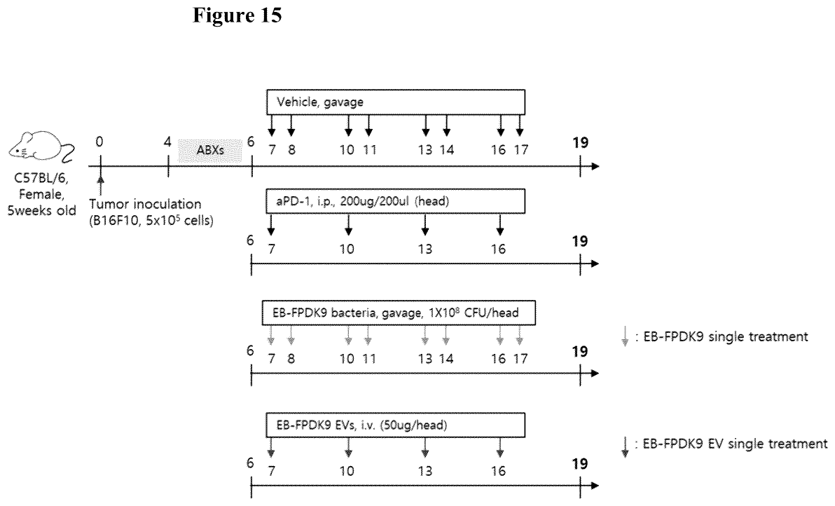

From day 7 after tumor inoculation, an anti-PD-1 cancer chemotherapeutic agent (aPD-1) as a positive control was intraperitoneally administered to each mouse once every 3 days at a concentration of 200 μg/200 μl. Meanwhile, the Faecalibacterium prausnitzii EB-FPDK9 strain was administered orally to each mouse twice every 3 days at 1×10⁸ CFU, and 50 μg of the Faecalibacterium prausnitzii EB-FPDK9 EVs were administered intravenously to each mouse (see FIG. 15).

The tumor size was measured using a ruler once every 3 days, and the tumor size was calculated according to the following equation: Tumor volume (mm³)=[major diameter×minor diameter²]/2.

### 7.2 Experimental Results

From day 7 after inoculation of the B16F10 cell line, the tumor size was measured and monitored, and on day 19, the mice were sacrificed and the tumor weight was measured. It was confirmed that, from day 13, tumor growth was inhibited in the group to which the positive control aPD-1 was administered and the groups to which each of the Faecalibacterium prausnitzii EB-FPDK9 strain and the Faecalibacterium prausnitzii EVs were administered, compared to tumor growth in the normal control group.

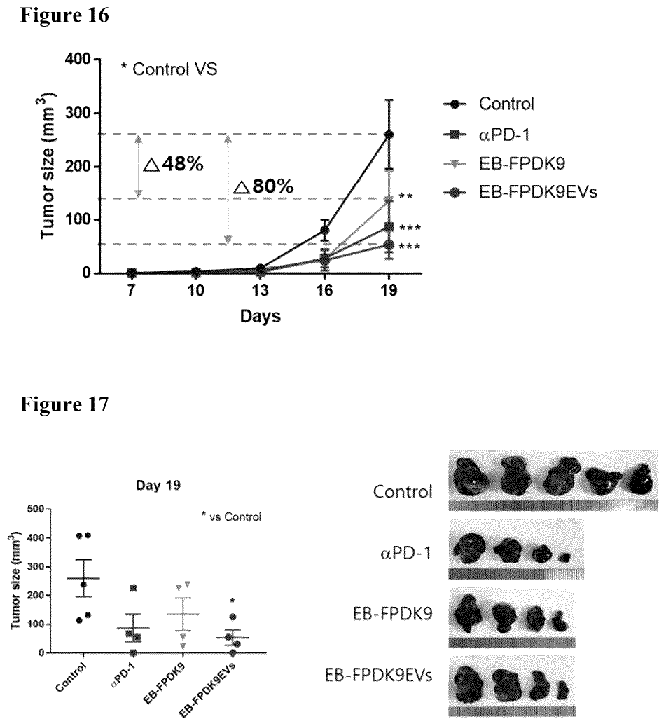

Referring to FIG. 16, it was confirmed that, on day 19 when the mice were sacrificed, the tumor size in the normal control group was 260.4±64.33 mm³, whereas the tumor size in the EB-FPDK9-administered group statistically significantly decreased to 135.6±56.46 mm³ (about 48% decrease) and the tumor size in the EB-FPDK9 EVs-treated group statistically significantly decreased to 53.41±26.51 mm³ (about 80% decrease).

Referring to FIG. 17, it was confirmed that the tumor weight significantly decreased in the group to which the positive control aPD-1 was administered and the group to which the Faecalibacterium prausnitzii EB-FPDK9 strain or the Faecalibacterium prausnitzii EB-FPDK9 EVs were administered, compared to tumor growth in the normal control group, and particularly, the tumor weight statistically significantly decreased in the group to which the Faecalibacterium prausnitzii EB-FPDK9 EVs were administered.

It was observed that the tumor size and weight in the group to which the Faecalibacterium prausnitzii EB-FPDK9 strain or the Faecalibacterium prausnitzii EVs were administered were similar to those in the aPD-1-administered group (see FIGS. 16 and 17). This demonstrates that administration of the Faecalibacterium prausnitzii EB-FPDK9 strain or the Faecalibacterium prausnitzii EVs alone exhibits anticancer efficacy comparable to that of aPD-1.

The embodiments disclosed herein are only illustrative of preferred embodiments and are not intended to limit the scope of the present invention. It will be apparent to those skilled in the art that the present invention may be modified or altered in various forms without departing from the spirit and scope thereof. The scope of protection of the present invention should be defined by the appended claims, and the above modifications and variations are intended to fall within the scope of protection of the present invention.

### Microorganism Deposit Accession Number

Depository authority: Korea Research Institute of Bioscience and Biotechnology

Accession number: KCCM12619P (EB-FPDK3)

Deposit date: Nov. 1, 2019

Depository authority: Korea Research Institute of Bioscience and Biotechnology

Accession number: KCCM12620P (EB-FPDK9)

Deposit date: Nov. 1, 2019

Depository authority: Korea Research Institute of Bioscience and Biotechnology

Accession number: KCCM12621P (EB-FPDK11)

Deposit date: Nov. 1, 2019

Depository authority: Korea Research Institute of Bioscience and Biotechnology

Accession number: KCCM12622P (EB-FPYYK1)

Deposit date: Nov. 1, 2019

1. A composition containing: isolated extracellular vesicles (EVs) from a Faecalibacterium sp. strain; and a pharmaceutically acceptable carrier or excipient.

2. The composition according to claim 1, wherein the Faecalibacterium sp. strain is a Faecalibacterium prausnitzii strain.

3. The composition according to claim 1, wherein the Faecalibacterium sp. strain is a Faecalibacterium prausnitzii EB-FPDK3 strain (KCCM12619P), an F. prausnitzii EB-FPDK9 strain (KCCM12620P), an F. prausnitzii EB-FPDK11 strain (KCCM12621P), or an F. prausnitzii EB-FPYYK1 strain (KCCM12622P).

4. The composition according to claim 1, wherein the extracellular vesicles (EVs) derived from the Faecalibacterium sp. strain have an average diameter of 20 to 300 nm.

5. (canceled)

6. The composition according to claim 1, wherein the composition is a pharmaceutical composition and the composition further contains a cancer chemotherapeutic agent and a cancer immunotherapeutic agent.

7. The composition according to claim 6, wherein the cancer immunotherapeutic agent is at least one selected from the group consisting of anti-PD1, anti-PDL1, anti-CTLA, anti-Tim3, and anti-LAG3.

8. The composition according to claim 6, wherein the extracellular vesicles (EVs) from the Faecalibacterium sp. strain and the cancer chemotherapeutic agent and the cancer immunotherapeutic agent are administered simultaneously in a single dosage form, or administered simultaneously or sequentially in separate dosage forms.

9. A pharmaceutical composition containing: an isolated Faecalibacterium sp. strain; and a pharmaceutically acceptable carrier or excipient.

10. The pharmaceutical composition according to claim 9, wherein the Faecalibacterium sp. strain is alive, pasteurized or heat-killed.

11. The pharmaceutical composition according to claim 9, wherein the Faecalibacterium sp. strain is a Faecalibacterium prausnitzii strain.

12. The pharmaceutical composition according to claim 9, wherein the Faecalibacterium sp. strain is a Faecalibacterium prausnitzii EB-FPDK3 strain (KCCM12619P), an F. prausnitzii EB-FPDK9 strain (KCCM12620P), an F. prausnitzii EB-FPDK11 strain (KCCM12621P), or an F. prausnitzii EB-FPYYK1 strain (KCCM12622P).

13. The composition according to claim 1, which is a pharmaceutical composition, a food composition, or a veterinary composition.

14. (canceled)

15. The composition according to claim 1, wherein the composition is an oral preparation in a form of powders, granules, tablets, capsules, suspensions, emulsions, syrups, or aerosols; a preparation for external use; a suppository, or a sterile injectable solution.

16. A method for preventing or treating cancer in a subject in need thereof, said method comprising administering an effective amount of a composition comprising bacterial extracellular vesicles (EVs) to the subject, wherein the bacterial EVs are from a Faecalibacterium sp. strain, and a pharmaceutically acceptable carrier or excipient.

17. The method according to claim 16, wherein the Faecalibacterium sp. strain is a Faecalibacterium prausnitzii strain selected from a Faecalibacterium prausnitzii EB-FPDK3 strain (KCCM12619P), an F. prausnitzii EB-FPDK9 strain (KCCM12620P), an F. prausnitzii EB-FPDK11 strain (KCCM12621P), or an F. prausnitzii EB-FPYYK1 strain (KCCM12622P).

18. The method according to claim 16, wherein the bacterial extracellular vesicles (EVs) have an average diameter of 20 to 300 nm.

19. The method according to claim 16, wherein the cancer is any one selected from the group consisting of colorectal cancer, lung cancer, small cell lung cancer, gastric cancer, liver cancer, blood cancer, bone cancer, pancreatic cancer, skin cancer, head or neck cancer, skin or intraocular melanoma, uterine cancer, ovarian cancer, rectal cancer, perianal cancer, colon cancer, breast cancer, fallopian tube carcinoma, endometrial carcinoma, cervical cancer, vaginal cancer, vulvar carcinoma, Hodgkin's disease, esophageal cancer, small intestine cancer, endocrine adenocarcinoma, thyroid cancer, parathyroid cancer, adrenal cancer, soft tissue sarcoma, urethral cancer, penis cancer, prostate cancer, chronic or acute leukemia, lymphocytic lymphoma, bladder cancer, kidney cancer, ureteral cancer, renal cell carcinoma, renal pelvic carcinoma, CNS tumor, primary CNS lymphoma, spinal cord tumor, brainstem glioma, and pituitary adenoma.

20. The method according to claim 16, which further comprising administering a cancer chemotherapeutic agent and a cancer immunotherapeutic agent to the subject.

21. The method according to claim 22, wherein the cancer immunotherapeutic agent is at least one selected from the group consisting of anti-PD1, anti-PDL1, anti-CTLA, anti-Tim3, and anti-LAG3.

22. The method according to claim 16, wherein the subject is human or non-human animal.
